(19) United States
(12) Patent Application Publication
(10) Pub. No.: US 2019/0269884 A1
(43) Pub. Date: Sep. 5, 2019

(54) CATHETER SYSTEM WITH REINFORCED GUIDEWIRE SHAFT AND METHOD OF MANUFACTURE

(71) Applicant: Medinol Ltd., Tel Aviv (IL)

(72) Inventor: Ben SAGI, Ramat Gan (IL)

(21) Appl. No.: 15/911,851

(22) Filed: Mar. 5, 2018

(51) Int. Cl. A61M 25/00; A61M 25/10; A61L 29/04; A61L 29/02; B32B 1/08; B32B 15/08; B32B 15/18; B32B 15/20; B32B 27/08; B32B 27/34; B32B 27/32; B32B 3/00; B29C 47/00; B29C 47/06; B29C 70/30

(52) U.S. Cl. CPC A61M 25/005; B29K 2027/18; A61M 25/0012; A61L 29/049; A61L 29/02; B32B 1/08; B32B 15/08; B32B 15/18; B32B 15/20; B32B 27/08; B32B 27/34; B32B 27/322; B32B 3/00; B29C 47/0045; B29C 47/0026; B29C 47/065; B29C 47/0004; B29C 70/30; B32B 2250/04; B32B 2305/08; B32B 2327/18; B32B 2377/00; B32B 2535/00; A61M 25/10

(57) ABSTRACT

The present invention relates to a catheter system having a reinforced guidewire shaft and a method of manufacturing a reinforced catheter shaft. In particular, the present invention relates to a catheter shaft having two or more polymer layers and a reinforcement layer that is comprised of braids or a coil or combination thereof. The braided reinforcement layer may have a constant picks per inch (PPI) between braids or a variable PPI between braids. Similarly, the coiled reinforcement layer may have a constant pitch space between coils or a variable pitch space between coils. The reinforced catheter shaft may be manufactured by a continuous reel-to-reel process using liquid polymers that are heat-hardened or by a discrete process using extruded tube that is shrunk with heat.

### FIELD OF THE INVENTION

The invention relates to a catheter system having a reinforced catheter shaft and a method of manufacturing a reinforced catheter shaft. In particular, the invention relates to a catheter shaft having a reinforcement layer comprised of braids or a coil or combination thereof. The braided reinforcement layer may have a constant picks per inch (PPI) or a variable PPI along the shaft. Similarly, the coiled reinforcement layer may have a constant pitch between coils or a variable pitch between coils. The reinforced catheter shaft may be manufactured by a continuous reel-to-reel process using liquid polymers that are heat-hardened or by a discrete process using extruded tube that is shrunk with heat.

### BACKGROUND

Conventional catheter systems, such as the catheters used in stent and balloon dilatation systems, are widely used during vascular procedures, for example, balloon angioplasty or stenting, for treating vascular disease. These catheters require a high degree of deliverability to properly reach the target location in the blood vessel. Two major aspects of deliverability of a catheter are pushability and flexibility. Pushability is defined as the ability to transmit pushing power from the proximal end of the catheter (held by the operator's hand) to the distal end of the catheter, for example, in order to traverse and cross calcifications, clots, occlusions, narrowed blood vessels, and other obstacles encountered during a vascular procedure. Flexibility is defined as the catheter's ability to bend, for example, during navigation through torturous vessels.

Catheter systems are typically inserted into a blood vessel over a flexible guidewire, which is inserted and navigated through the vessel before insertion of the catheter. During the procedure, the catheter slides over the guidewire through a guidewire shaft—a tube running through the length of the catheter. In some catheter systems, the guidewire shaft runs through the entire length of the catheter while in other systems it may run only in the more distal part of the catheter. In any event, friction between the guidewire and the shaft would reduce pushability and affect flexibility of the catheter thus reducing overall deliverability.

Sometimes, while using a cardiovascular catheter, the effective diameter of the shaft lumen is reduced or the generally circular shape of the shaft lumen changes shape (e.g., becomes more ellipsoidal), and may create or increase the friction between the shaft (more particularly, the inner wall of the catheter) and the guidewire. For example, FIG. 1A illustrates a side view of a straight catheter shaft 100a and the corresponding generally circular cross-sectional shape of the straight catheter shaft 100a having diameter D₁. Upon bending the straight catheter shaft 100b, as illustrated in FIG. 1B, the cross-sectional shape of the catheter shaft 100b becomes ellipsoidal having smaller diameter D₂ and larger diameter D₃ where the smaller diameter D₂ of the ellipsoidal shape may cause friction against the guidewire. As a result, the friction between the inner wall and guidewire may reduce overall deliverability of the catheter system—either as the catheter traverses a vessel before the procedure, for example, balloon angioplasty or stenting, or while pulling out the catheter after the procedure. This may happen for instance while navigating the catheter through a sharp curve in a blood vessel lumen, causing the cross section of the guidewire shaft to become less circular and more ellipsoid in shape.

Additionally, during balloon inflation in procedures such as balloon angioplasty and stenting, the fluid pressure used to inflate the balloon at the end of the catheter may cause longitudinal elongation of the shaft, thus reducing the diameter of the guidewire lumen. For example, FIG. 2A illustrates a straight catheter shaft 200 having an uninflated balloon 210a and a generally uniform diameter D₁ along the length of the catheter shaft 200a. Upon inflation of the balloon 210b, as illustrated in FIG. 2B, the catheter shaft 200b is collapsed at the region within the inflated balloon 210b causing diameter D₂ of the catheter shaft 200b to decrease. The decreased diameter D₂ of the catheter shaft 200b may cause friction on the guidewire. In any case, friction or other forces affecting the guidewire shaft and the guidewire may increase during balloon inflation or during catheter insertion or retraction from the body.

One possible solution to this requirement may be to increase the diameter of the guidewire shaft. However, that would increase the catheter's crossing profile and reduce deliverability. Another solution may be coating the inner wall of the guidewire shaft with materials having low friction coefficient such as Teflon, HDPE (high density polyethylene), etc. This as well may increase the crossing profile of the catheter and thus reduce deliverability.

Thus, in view of the foregoing, there exists a need to reduce or prevent friction between the guidewire and the guidewire shaft (i.e., the inner wall of the catheter) during vascular procedures. In particular, there exists a need for reducing or preventing friction between the guidewire and the guidewire shaft while maintaining a small crossing profile as possible for the catheter.

### SUMMARY OF THE INVENTION

A catheter shaft of the present invention comprises a plurality of polymer layers and a reinforcement layer extending through all or a portion of the catheter shaft. The reinforcement layer may comprise a braided or coiled tubular structure or a combination of both. The picks per inch (PPI) in the case of a braided reinforcement layer or the pitch in the case of a coiled reinforcement layer may vary along the shaft or may be constant.

The distal tip of the catheter may be tapered or have a constant diameter with the rest of the catheter. The catheter further comprises an inner polymer layer and one or more additional layers. The distal tip of the catheter may include the reinforcement layer or may consist of only one or more polymer layers to form an atraumatic tip.

### BRIEF DESCRIPTION OF THE DRAWINGS

The present invention will be understood and appreciated more fully from the following detailed description taken in conjunction with the drawings in which:

FIG. 1A shows a straight shaft lumen and a cross section of the straight shaft lumen.

FIG. 1B shows a shaft lumen during bending and a cross section of the shaft lumen during bending, illustrating the problem in the prior art.

FIG. 2A shows an uncompressed shaft lumen before balloon inflation.

FIG. 2B shows a compressed shaft lumen after balloon inflation, illustrating the problem in the prior art.

FIG. 3A shows a catheter shaft having a braided reinforcement layer with a constant picks per inch (PPI) along the length of the catheter shaft.

FIG. 3B shows a catheter shaft having a braided reinforcement layer with a variable picks per inch (PPI) along the length of the catheter shaft.

FIG. 4A shows a catheter shaft having a coiled reinforcement layer with a variable pitch between adjacent coils.

FIG. 4B shows a catheter shaft having a coiled reinforcement layer with a variable pitch between adjacent coils and an atraumatic tip.

FIG. 5A shows a catheter shaft having a reinforcement layer including a coiled reinforcement section having a constant pitch and a braided reinforcement section having a constant PPI and an atraumatic tip.

FIG. 5B shows a catheter shaft having a reinforcement layer including a coiled reinforcement section having a constant pitch and a braided reinforcement section having a variable PPI.

FIG. 6 shows a distal tip of a catheter shaft having a tapered, atraumatic tip.

FIG. 7 shows a distal-most tip region of a catheter shaft in addition to three options for modifying the distal tip.

FIG. 8 shows a cross-sectional view of a catheter shaft having two polymer layers and a braided reinforcement layer.

FIG. 9 shows a cross-sectional view of a catheter shaft having three polymer layers and a braided reinforcement layer.

FIG. 10A shows a cutaway view of a catheter shaft having a braided reinforcement layer.

FIG. 10B shows a cross-sectional view of a catheter shaft having a braided reinforcement layer and an atraumatic tip.

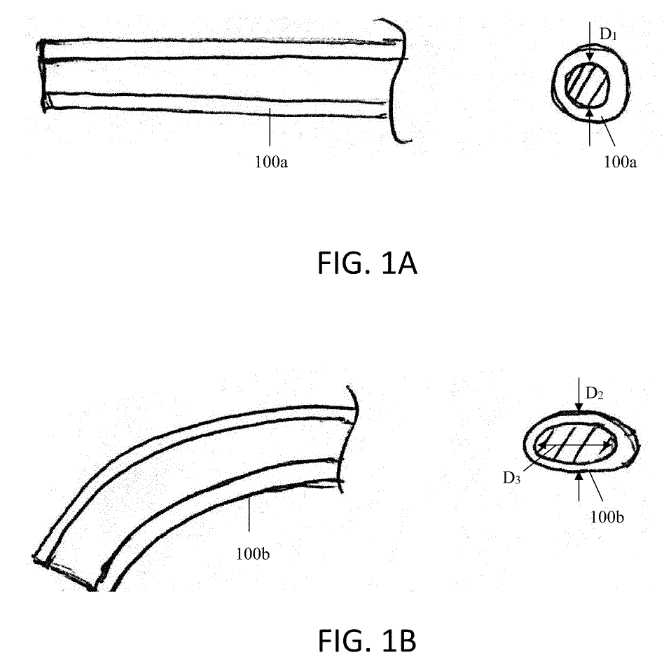

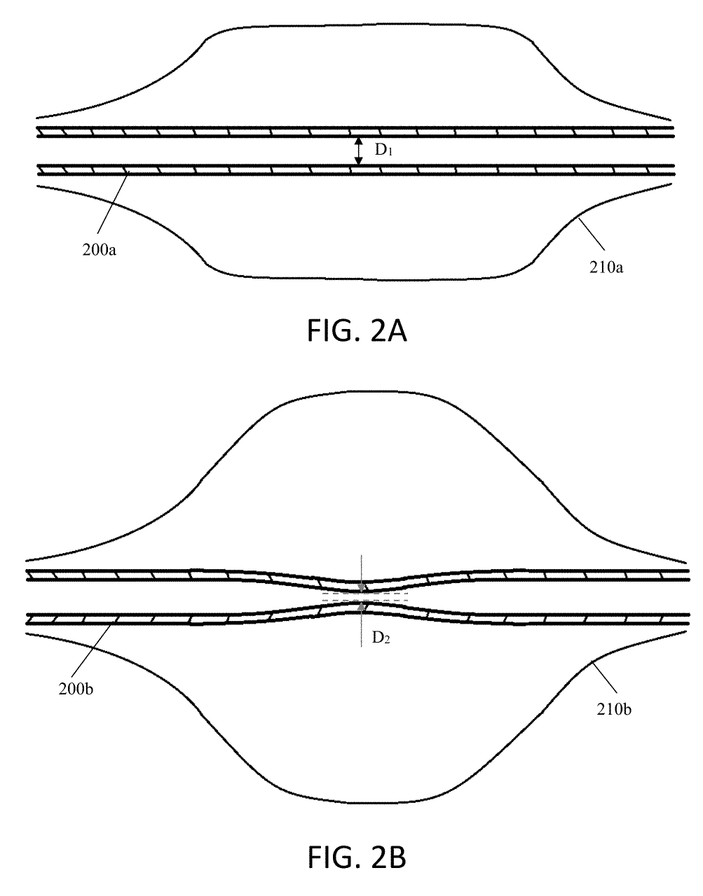

### DETAILED DESCRIPTION OF THE INVENTION

A catheter shaft of the invention generally includes a catheter wall having an inner layer, an outer layer, and a reinforcement layer disposed between the inner and outer layers. Alternatively, the catheter wall may include one or more middle layers in addition to the inner layer, outer layer, and reinforcement layer. Deliverability properties (i.e., pushability and flexibility) of the catheter may be modified by changing the material(s) of the reinforcement layer, the material(s) of the inner layer, the middle layer(s), or the outer layer, the longitudinal (i.e., along the length of the catheter shaft) density of the reinforcement layer (e.g., pitch or PPI), and/or the structure of the reinforcement layer (e.g., a braid and/or a coil). The inner layer of the catheter shaft is defined as the layer that creates the inner luminal surface of the catheter shaft lumen while the outer layer of the catheter shaft is defined as the layer directly abutting a vessel wall and the middle layer (if included) comprises the one or more layers between the inner layer and the outer layer.

In one embodiment, the catheter shaft may include more than two polymer layers. For example, the catheter wall may include multiple polymer layers, e.g., three, four, or five layers of polymers, although one skilled in the art will recognize that any suitable number of layers may be used to form the catheter shaft. In any case, each layer may include the same polymer or a different polymer.

The catheter shaft may further include a balloon coupled to a distal end of the outer wall at a bonding region. The balloon may be secured to the catheter wall by, for example, adhesives or other suitable polymer-to-polymer bonding as is known in the art.

The catheter shaft may further include a catheter tip at the distal end of the catheter shaft. The catheter tip may extend beyond the bonding region by about 0.3 mm to 10 mm. In one embodiment, the catheter tip may include a distal taper, tapering from a first diameter to a second, smaller diameter at the distal-most end of the catheter shaft. The catheter tip may taper from the balloon bonding region to a distal-most end of the catheter shaft. In another embodiment, the longitudinal length of the catheter shaft may have a uniform diameter along its entire length. In this embodiment, the distal-most end may have a rounded edge. In another embodiment, the distal tip may include a tapered outer diameter and a tapered inner diameter. For example, the outer diameter and the inner diameter may both taper at the same rate. In another example, the outer diameter may include a taper at a first rate while the inner diameter tapers at a second rate.

The reinforcement layer of the catheter shaft may comprise a braided reinforcement layer and/or a coiled reinforcement layer disposed between the inner layer and the outer layer of the catheter. In embodiments having more than two layers, the reinforcement layer may be disposed between any two adjacent layers in the catheter shaft. The reinforcement layer may extend to the distal-most end of the catheter shaft. Alternatively, the braided reinforcement layer or coiled reinforcement layer may terminate proximal to the distal-most end, leaving a portion of the catheter tip made entirely of one or more layers of polymer as an atraumatic tip. The atraumatic tip may have a length between 0.3 mm and 10 mm.

In the embodiment where the catheter shaft includes a taper on either or both of the inner diameter and the outer diameter, the reinforcement layer (either the coil or the braided reinforcement layer) may also have a taper. For example, the reinforcement layer may have a first, constant diameter along a first length of the catheter shaft and taper to a second, smaller diameter along a second length that is distal to the first length.

The reinforcement layer of the catheter shaft may include a braided reinforcement structure/layer. A braided reinforcement layer includes crisscrossing wires that are woven around the circumference of the catheter wall. The braided reinforcement layer may include a picks per inch (PPI) of about 20 to about 150. PPI is defined as the number of weft threads of wire per inch of woven braid. A higher PPI value corresponds to a higher number of windings per inch of the catheter wall and a more flexible catheter wall. A lower PPI value corresponds to a lower number of windings per inch of the catheter wall and a corresponding less flexible catheter wall. In an example, the braided reinforcement layer may have a constant PPI along the entire length of the catheter wall. In an alternative example, the braided reinforcement layer may have a variable PPI along the length of the catheter. The braided reinforcement layer may include any suitable braid as is known in the art. For example, the braided reinforcement layer may include a “1 under 2 over 2” braid. Other suitable braids may include “over 1 under 1,” “over 2 under 2,” a diamond pattern, or a variable pick.

The reinforcement layer of the catheter shaft may include a coiled reinforcement structure/layer. A coiled reinforcement layer includes one or more coiled wires around the circumference of the catheter wall. The coiled reinforcement layer may include a pitch (i.e., a distance between adjacent coils) of about 0.1 mm to about 0.5 mm. A lower pitch corresponds to a higher number of windings per inch of the catheter wall and a more flexible catheter wall. A higher pitch corresponds to a lower number of windings per inch of the catheter wall and a less flexible catheter wall. In an example, the coiled reinforcement layer may have a constant pitch between adjacent windings along the entire catheter length. In another, the coiled reinforcement layer may have a variable pitch along the catheter length. As an example of a coiled reinforcement layer having variable pitch, the coiled reinforcement layer may gradually increase in pitch from a proximal end to a distal end of the catheter wall or the coiled reinforcement layer may gradually decrease in pitch from a proximal end to a distal end of the catheter wall. At the distal-most end of the catheter shaft, the coiled reinforcement layer may be wound into a circular ring so as not to leave a free end of wire.

The reinforcement layer of the catheter shaft may be a hybrid of the two reinforcement layers described above, such that the reinforcement layer includes both a coiled reinforcement layer region and a braided reinforcement layer region. In one embodiment, the coiled reinforcement region may be proximal to the braided reinforcement region. In an alternative embodiment, the coiled reinforcement region may be distal to the braided reinforcement region. Each of the coiled reinforcement region and braided reinforcement region may provide different deliverability characteristics. For example, the coiled reinforcement region may be more flexible in bending but less resistant to an axial load (e.g., pulling forces or pushing forces) when compared to the braided reinforcement region. Each of the coiled reinforcement region and braided reinforcement region may have any suitable length along the length of the catheter wall. The preferred length for the coiled portion is about 3 mm to 300 mm and the preferred length for the braided portion is about 150 mm to 300 mm. The interface region between the coiled reinforcement region and braided reinforcement region can be located anywhere between the proximal end of the catheter and the distal tip.

Generally, the inner layer of the catheter shaft may include a thickness of 0.025 mm to 0.5 mm, preferably about 0.13 mm. The optional middle layer(s) may include a thickness of 0.025 mm to 0.5 mm, preferably about 0.06 mm. The outer layer may include a thickness of 0.025 mm to 0.5 mm, preferably about 0.04 mm.

The inner layer of the catheter shaft may comprise a polymer that has a low coefficient of friction so as to minimize any friction against a guidewire when the catheter shaft is inserted over the guidewire. For example, the inner layer may be made of expanded polytetrafluoroethylene (ePTFE or Teflon) or any other suitable polymer as is known in the art, preferably one having a low frictional coefficient.

The middle and outer layers may be made of the same or different biocompatible material. For example, the middle and outer layers may be made from polyether block amide (PEBAX), polyamide, polypropylene, polyether ether ketone (PEEK), polyimide (PI), polyolefins (e.g., polypropylene, polyethylene) or other suitable materials as is known in the art.

The braided reinforcement layer and/or the coiled reinforcement layer may comprise one or more metals or metal alloys. For example, the braided reinforcement layer and/or the coiled reinforcement layer may be made of stainless steel, titanium, gold, cobalt-chromium, platinum-iridium, nitinol, an amorphous metal alloy and/or any suitable combination of metals or metal alloys. Alternatively, the braided reinforcement layer and/or the coiled reinforcement layer may be made of a polymer. For example, the braided reinforcement layer and/or the coiled reinforcement layer may be made of polyamide, nylon, polyurethane, poly-paraphenylene terephthalamide, polyether ether ketone (PEEK), or any suitable biocompatible polymer as is known in the art.

The individual wires of the braided reinforcement layer and/or the coiled reinforcement layer may have a width of 0.0002 inch (0.00508 mm) to 0.01 inch (0.254 mm) and may have any suitable cross section, e.g., rounded or rectangular. One of skill in the art will recognize that any suitable width and diameter of wire may be used for the reinforcement layer.

As the catheter shaft bends and/or twists during insertion or retraction from a blood vessel (or other lumen), the braided reinforcement layer and/or the coiled reinforcement layer prevents the catheter wall from collapsing to a reduced diameter causing friction against the guidewire. Moreover, during inflation of a balloon, the braided reinforcement layer and/or the coiled reinforcement layer prevents the catheter wall from stretching, deforming, and/or shrinking due to the higher fluid pressure around the catheter wall in the balloon region. Stretching, deforming, and/or shrinking of the catheter wall may reduce the diameter of the catheter wall and thus also contribute to friction against the guidewire.

In a preferred example, the balloon may be secured to the outer wall by heat welding to fuse polymer of the balloon with polymer of the outer layer of the catheter shaft. Heat welding may include contacting a heated die to a portion of the balloon and the outer layer of the catheter wall at a balloon bonding region.

Optionally, the distal tip of the catheter may include an atraumatic tip comprising a taper. The atraumatic tip may include any of the same polymers that comprise the catheter shaft as described above such as, for example, polyimide (PI), polyamide (PA), or PEBAX. The distal tip may be tapered by reducing the diameter of the outer layer by, for example, machining the outer layer using a lathe. The distal tip may be tapered using a thermal process including a heated die having the desired tapered shape. In particular, the steps of tapering the distal tip include: positioning a heat shrink tube around the distal tip of the catheter, positioning the distal tip (having the heat shrink tube thereon) within a heated die, and closing the heated die over the distal tip to form the desired tapered shape. As the die closes on the tip, the combination of heat, die geometry, and the heat shrink tube form the distal tip into the desired tapered shape.

In another embodiment, the distal tip of the catheter may include a rounded, non-tapered tip. The rounded, non-tapered tip may be formed by positioning the distal tip in a heated die having the desired tip shape and end finish. The heated die is closed such that the polymer at the distal tip is pressed and formed into the desired rounded, non-tapered shape.

The following examples may incorporate one or more of the features described above. Said examples do not limit the invention but serve to illustrate exemplary embodiments within the scope of the invention.

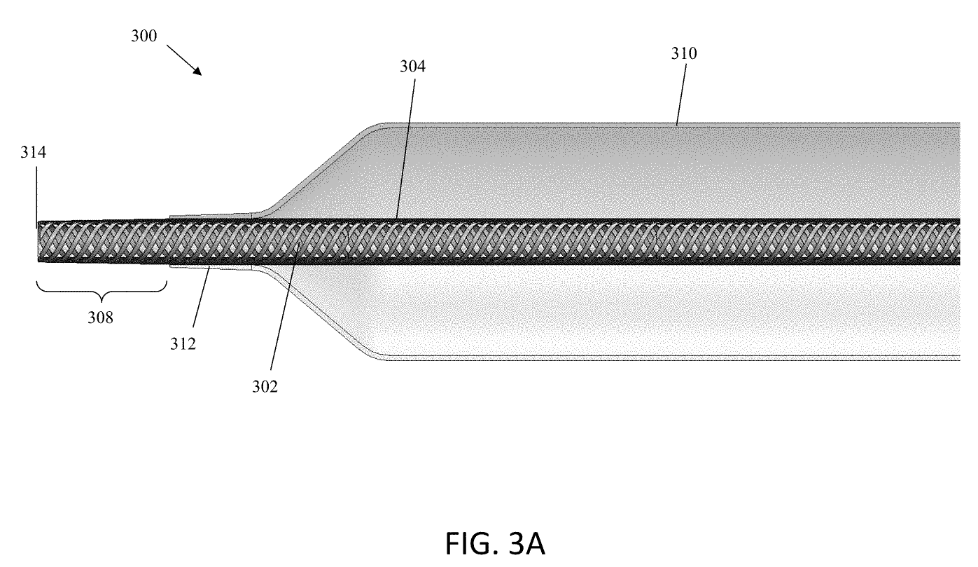

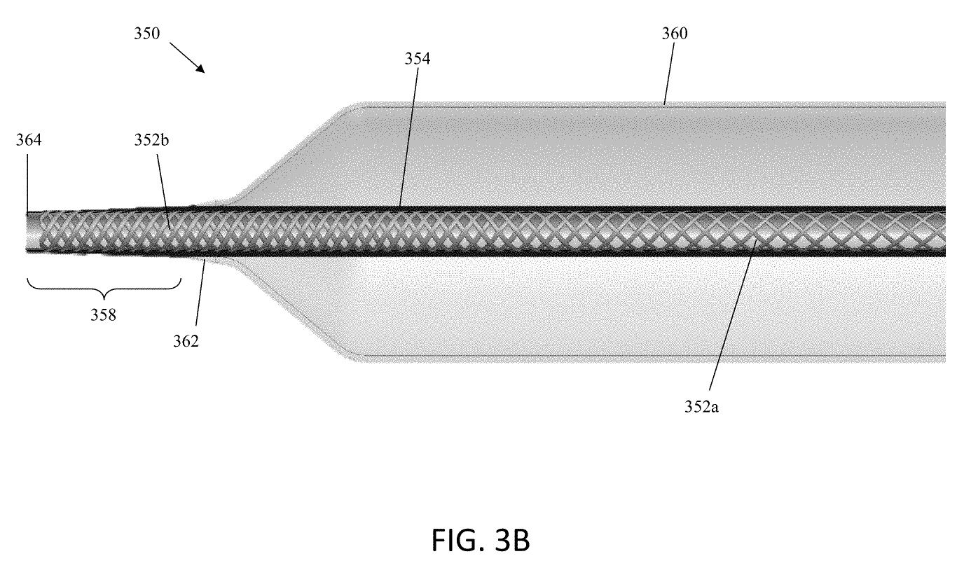

FIG. 3A shows a catheter shaft 300 having a braided reinforcement layer 302 with a constant picks per inch (PPI) along the length of the catheter shaft 300. The catheter shaft 300 includes a tubular catheter wall 304 that may include multiple layers of polymers (e.g., two, three, four, five, or more layers). In an example, the catheter wall 304 includes an inner layer and an outer layer. Between the inner and outer layers of polymer, the catheter wall 304 includes a reinforcement layer 302 that extends substantially the entire length of the catheter wall 304. The reinforcement layer 302 may extend for a portion of the length of the catheter wall 304.

A braided reinforcement layer 302 includes crisscrossing wires woven around the circumference of the catheter wall 304. In FIG. 3A, the braided reinforcement layer 302 has a constant PPI along the entire length of the catheter wall 304.

As described above, the braided reinforcement layer 302 is disposed between polymer layers of the catheter wall 304. In one embodiment, the braided reinforcement layer 302 may be embedded within a layer of the catheter wall 304 (e.g., if a continuous process of manufacturing is used as described below).

The catheter shaft 300 of FIG. 3A further includes a balloon 310 coupled to a distal portion of the catheter wall 304 at a bonding region 312. FIG. 3A illustrates the balloon 310 secured to the outer layer of the catheter wall 304. The balloon 310 is secured to the catheter wall 304 by, for example, adhesives or other suitable polymer-to-polymer bonding.

FIG. 3A further illustrates a catheter tip 308 at the distal end of the catheter shaft 300. As shown in FIG. 3A, the catheter tip 308 includes a distal taper, i.e., tapering from a first diameter (of the catheter wall 304) to a second, smaller diameter (at the distal-most end 314). The catheter tip 308 may taper from the balloon bonding region 312 to a distal-most end 314 of the catheter shaft 300.

FIG. 3B shows a catheter shaft 350 having braided reinforcement layer regions 352a and 352b with a variable picks per inch (PPI) between adjacent braids along the length of the catheter shaft 350. Similar to FIG. 3A above, the catheter shaft 350 includes a catheter wall 354 that comprises two or more layers, a balloon 360 coupled to the catheter wall 354 at the balloon bonding region 362, and a catheter tip 358 at the distal end of the catheter shaft 350 having a distal taper to a distal-most end 364 of the catheter shaft 350.

Similar to the braided reinforcement layer of FIG. 3A, the proximal reinforcement region 352a and distal reinforcement region 352b each include crisscrossing wires that are woven around the circumference of the catheter wall 354. The proximal reinforcement region 352a and distal reinforcement region 352b are contiguous and together extend substantially the entire length of the catheter shaft 350. In FIG. 3B, the reinforcement regions 352a and 352b have a varying PPI along the length of the catheter wall 354. For example, the proximal reinforcement region 352a has a lower PPI that gradually becomes higher towards the distal reinforcement region 352b at the distal most end 364 of the catheter wall 354. In the embodiment shown in FIG. 3B, the catheter wall 354 has lower flexibility (i.e., stiffer) at the more proximal end of the catheter wall 354 and higher flexibility at the distal end of the catheter wall 354.

In another embodiment (not shown), the proximal reinforcement region may have a higher PPI than the distal reinforcement region. The resulting catheter shaft is more flexible in the proximal reinforcement region than the distal reinforcement region. Thus, the catheter wall may be more flexible at the proximal end of the catheter wall and stiffer at the distal end of the catheter wall in this embodiment.

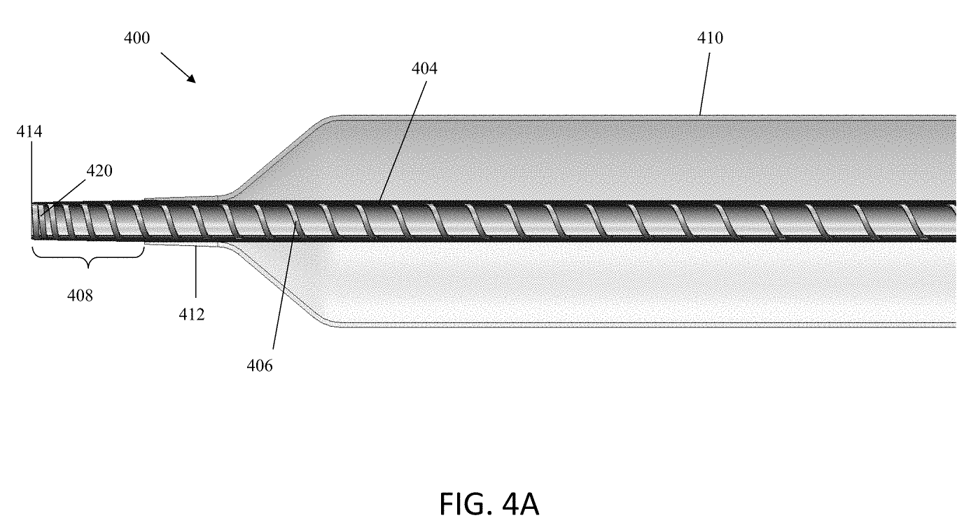

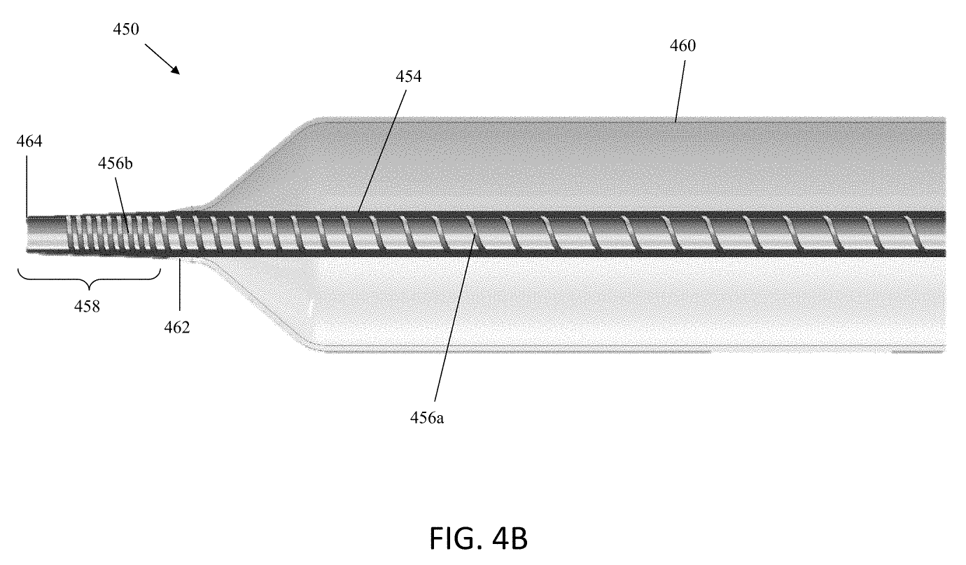

FIG. 4A shows a catheter shaft 400 having a coiled reinforcement layer 406 with a variable pitch along the length of the catheter shaft 400. In FIG. 4A, the pitch of the coiled reinforcement layer 406 decreases towards the distal end of the catheter shaft 400. Similar to FIGS. 3A and 3B, the catheter shaft 400 includes a catheter wall 404 comprising two or more polymer layers, a balloon 410 coupled to the catheter wall 404 at the balloon bonding region 412, and a catheter tip 408 at the distal end of the catheter shaft 400 having a distal taper to a distal-most end 414 of the catheter shaft 400.

The coiled reinforcement layer 406 includes a coiled wire around the circumference of the catheter wall 404. In FIG. 4A, the coiled reinforcement layer 406 has a variable pitch between adjacent windings with a larger pitch towards the proximal end of the catheter shaft 400 and a smaller pitch towards the distal end of the catheter shaft 400. At the distal-most end 414 of the catheter shaft 400, an end 420 of the coiled reinforcement layer 406, e.g., the last 2-3 loops, may be wound into a circular ring so as not to leave a free end of wire. For example, the last 2-3 loops of the coiled reinforcement layer 406 may be welded into a ring. Alternatively, a coil/marker band may be attached to the end 420. In yet another alternative embodiment, the end 420 can be left as an unfinished coil after the coil is cut.

FIG. 4B shows a catheter shaft 450 having proximal reinforcement region 456a and a distal reinforcement region 456b with a variable pitch between adjacent coils along the length of the catheter shaft 450. Similar to FIGS. 3A, 3B, and 4A, the catheter shaft 450 includes a catheter wall 454 comprising two or more layers, a balloon 460 coupled to the catheter wall 454 at the balloon bonding region 462, and a catheter tip 458 at the distal-most end of the catheter shaft 450 having a distal taper to a distal-most end 464 of the catheter shaft 450.

Similar to the coiled reinforcement layer of FIG. 4A, the proximal reinforcement region 456a and distal reinforcement region 456b each include wires that coil around the circumference of the catheter wall 454. The reinforcement regions 456a and 456b are contiguous and together extend substantially the entire length of the catheter shaft 450. In FIG. 4B, the reinforcement regions 456a and 456b have a variable pitch along the length of the catheter wall 454. For example, the proximal reinforcement region 456a has a higher pitch that gradually becomes smaller towards the distal reinforcement region 456b of the catheter wall 454. In the embodiment shown in FIG. 4B, the catheter wall 454 has lower flexibility (i.e., stiffer) in the proximal portion of the catheter wall 454 and higher flexibility in the distal portion of the catheter wall 454.

Alternatively (not shown), the proximal reinforcement may have a lower pitch than the distal reinforcement region. In this embodiment, the proximal reinforcement region is more flexible than the distal reinforcement region. In this embodiment, the resulting catheter wall will be more flexible at the proximal end of the catheter wall and stiffer at the more distal end of the catheter wall.

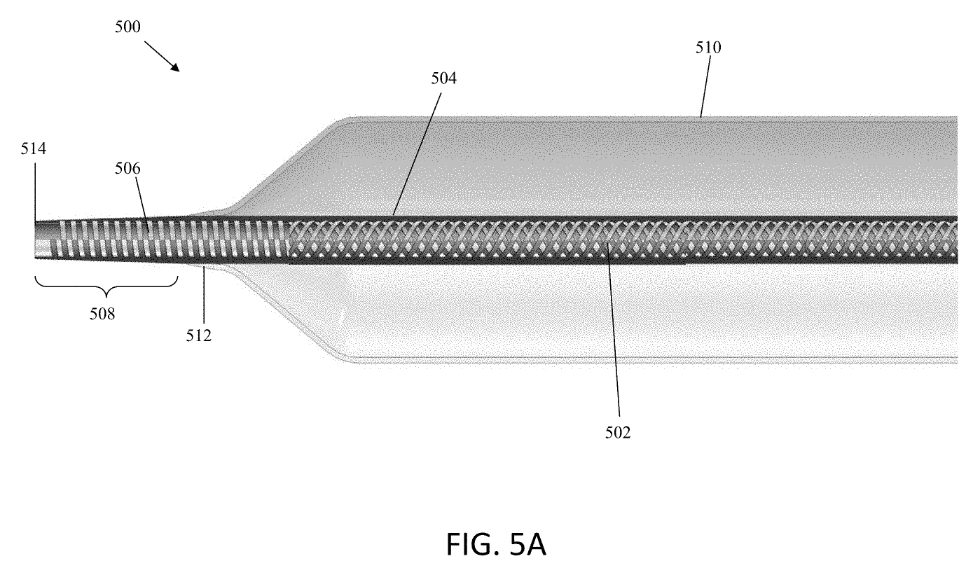

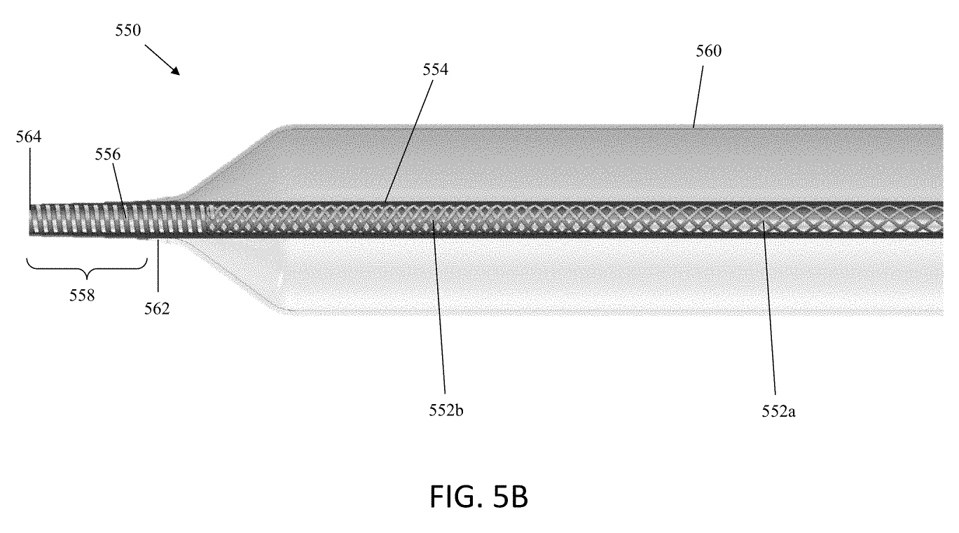

In some embodiments of the invention, combining the unique properties of a coiled reinforcement and a braided reinforcement in a single catheter shaft may provide additional utility to an operator. FIG. 5A shows a catheter shaft 500 having a reinforcement layer including a coiled reinforcement region 506 having a constant pitch and a braided reinforcement region 502 having a constant PPI. Similar to the preceding figures, the catheter shaft 500 includes a catheter wall 504 that may comprise two or more polymer layers, a balloon 510 coupled to the catheter wall 504 at the balloon bonding region 512, and a catheter tip 508 at the distal end of the catheter shaft 500 having a distal taper to a distal-most end 514 of the catheter shaft 500.

The catheter wall 504 of FIG. 5A includes both a coiled reinforcement region 506 and a braided reinforcement region 502 contiguous with one another. The coiled reinforcement region 506 and the braided reinforcement region may be coupled to one another or not coupled at all. In the embodiment wherein the coiled reinforcement region 506 and the braided reinforcement region are coupled, welding or a mechanical interlock may be used for the coupling. Alternatively, one of the braided coils may be continued without the other braided coils in order to maintain continuity of structure. The braided reinforcement region 502 may be substantially similar to the braided reinforcement region of FIG. 3A and the coiled reinforcement region 506 may be substantially similar to the coiled reinforcement region of FIG. 4A. In FIG. 5A, the coiled reinforcement region 506 includes a constant pitch between adjacent coils and the braided reinforcement region 502 includes a constant PPI.

In FIG. 5A, the coiled reinforcement region 506 includes a coiled wire that is wound around the circumference of the catheter wall 504 in the distal portion of the catheter shaft 500 and the braided reinforcement region 502 includes crisscrossing wires that are woven around the circumference of the catheter wall 504 in the proximal portion of the catheter shaft 500. As described above, each of the coiled reinforcement region 506 and braided reinforcement region 502 may provide different deliverability characteristics to the catheter shaft 500. In FIG. 5A, the distal end of the catheter shaft 500 will be more flexible while the braided reinforcement region 502 will provide better lumen stability to the overall catheter shaft 500.

As described above, the braided reinforcement region and coiled reinforcement region may have any suitable length. In FIG. 5A, the catheter wall 504 includes a braided reinforcement region 502 which extends along the length of the catheter wall 504 from a proximal end (e.g., closer to the operator) to a distal end that corresponds to a balloon 510 or a portion thereof (as shown), whereas the length of the catheter wall 504 corresponding to the length of the catheter tip 508 may include a coiled reinforcement region 506. As shown in FIG. 5A, the coiled reinforcement region 506 is distal to the braided reinforcement region 502 and extends from the distal tip of the catheter shaft through a portion of the balloon length. In another embodiment, the coiled reinforcement region 506 may be proximal to the braided reinforcement region 502.

FIG. 5B shows a catheter shaft 550 having a reinforcement layer including a coiled reinforcement region 556 having a constant pitch and braided reinforcement regions 552a and 552b having a variable PPI. The catheter shaft 550 includes a catheter wall 554 that includes two or more polymer layers, a balloon 560 coupled to the catheter wall 554 at the balloon bonding region 562, and a catheter tip 558 at the distal end of the catheter shaft 500 having a distal taper to a distal-most end 564 of the catheter shaft 550.

Similar to the catheter wall 504 of FIG. 5A, the catheter wall 554 includes both a coiled reinforcement region 556 and braided reinforcement regions 552a and 552b contiguous with one another. Similar to FIG. 5A, the coiled reinforcement region 556 may be coupled to the braided reinforcement region 552b or not coupled at all. In FIG. 5B, the coiled reinforcement region 556 includes a constant pitch between adjacent coils and the proximal reinforcement region 552a and distal reinforcement region 552b include a variable PPI between wefts of the braids.

The proximal reinforcement region 552a and distal reinforcement region 552b include a variable PPI that gradually increases from a proximal end of the catheter wall 554 to a distal end of the catheter wall 554. In an alternative embodiment, the PPI of the braided reinforcement region may gradually decrease from a proximal end of the catheter wall 554 to a distal-most end 564 of the catheter wall 554.

In FIG. 5B, the coiled reinforcement region 556 is distally adjacent to the distal reinforcement region 552b and extends from the distal tip to the balloon shoulder. The proximal braided reinforcement region 552a and distal braided reinforcement region 552b extend along substantially the entire longitudinal length of the balloon 560 and may further extend proximally to the proximal end of the catheter wall 554.

Each of the coiled reinforcement region 556 and proximal 552a and distal 552b reinforcement regions may have any suitable length along the length of the catheter wall 554. For example, the catheter wall 554 may include proximal 552a and distal 552b reinforcement regions along the length of the catheter shaft 550 that houses all or a portion of the balloon 560. The distal reinforcement region 552b has a higher PPI and may correspond to a location along the balloon 560 where the greatest forces are exerted on the catheter wall 554 during inflation because a higher PPI braided reinforcement will provide greater resistance to any deformation of the catheter wall 554. The portion of the catheter wall 554 corresponding to the catheter tip 558 may include a coiled reinforcement region 556 as the coiled reinforcement region may provide more flexibility than a braided reinforcement region. In an alternative embodiment (not shown), the coiled reinforcement region may be proximal to the reinforcement regions. For example, the catheter tip may include a braided reinforcement region while the proximal portion of the catheter wall corresponding to the balloon may include a coiled reinforcement region having either a constant pitch or a variable pitch.

In yet another embodiment, the coiled reinforcement region 556 may include a variable pitch between the coils. The coiled reinforcement region 556 may include proximal coils having a smaller pitch and distal coils having a larger pitch. In an alternative embodiment, the coiled reinforcement region 556 may include proximal coils having a larger pitch and distal coils having a smaller pitch.

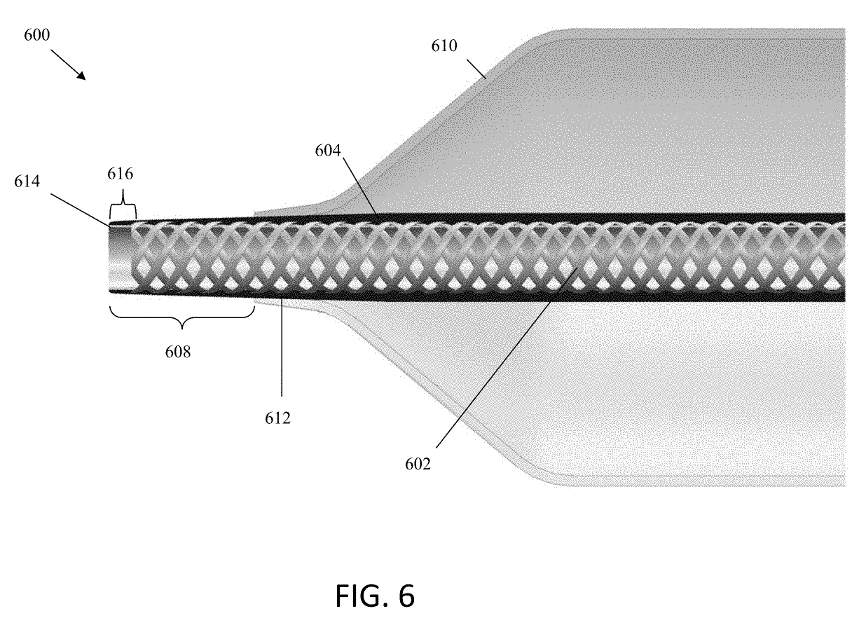

FIG. 6 shows a distal tip 608 of a catheter shaft 600. As shown in FIG. 6, the catheter tip 608 may extend distal to the bonding region 612 of the balloon 610. For example, the catheter tip may extend distal to the bonding region 612 by about 2.0 mm to 15.0 mm. As shown in the preceding figures, the catheter tip 608 may include a taper on the outer layer of the catheter wall 604 from the balloon bonding region 612 to the distal-most end 614 of the catheter shaft. The tapered outer layer may be achieved by machining the outer polymer layer of the catheter wall 604 to gradually remove polymeric material along the catheter wall 604 length to the distal-most end 614. The tapered catheter tip 608 may be useful as the taper provides for a reduced entry profile for the catheter shaft 600 when inserting the catheter shaft 600 into a blood vessel lumen.

The braided reinforcement layer 602 (and/or coiled reinforcement layer) may terminate proximal to a distal-most end 614, leaving a portion of the catheter tip 608 entirely made of one or more polymer layers as an atraumatic tip 616. FIGS. 3B, 4B, and 5A similarly show a reinforcement layer that terminates proximal to the distal-most end 614 of the catheter shaft, thus forming an atraumatic tip. The atraumatic tip may have a length of about 0.10 mm to 5.0 mm. Alternatively, as shown in FIGS. 3A, 4A, and 5B, embodiments not having an atraumatic tip have the braided reinforcement layer (and/or coiled reinforcement layer) extending to the distal-most end 614 of the catheter shaft.

In another embodiment (not shown), the catheter wall and catheter tip may have a uniform diameter along the entire length of the catheter shaft. In this embodiment, the distal-most end may have a rounded edge. The rounded distal most end may be formed by polishing the distal-most end until smooth.

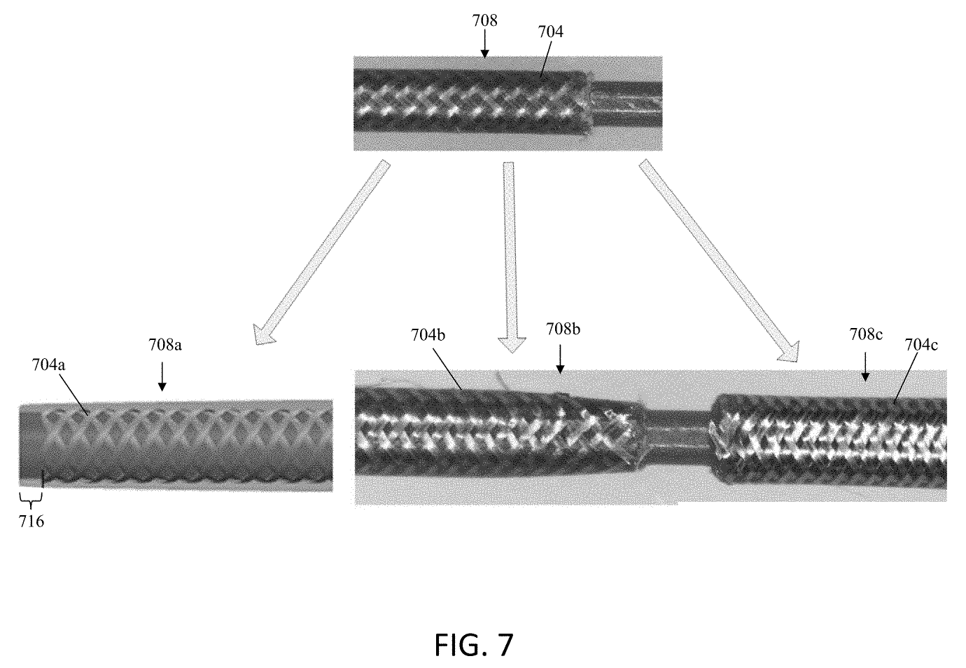

FIG. 7 shows a catheter distal-most tip region 708 of a catheter shaft in addition to three options 708a-708c for modifying the distal catheter tip 708. In one embodiment, catheter tip 708a includes a catheter wall 704a having a braided reinforcement layer (or coiled reinforcement layer) and a tapered atraumatic tip 716. In particular, the braided reinforcement layer (or coiled reinforcement layer) of catheter tip 708a terminates proximal to the distal most edge thus forming an atraumatic tip made entirely of polymer. In this embodiment, one or more of the inner layer, middle layer(s) (if included), and outer layer extend beyond the reinforcement layer to form the atraumatic tip. The atraumatic tip may be tapered or not tapered. In another embodiment, catheter tip 708b includes a catheter wall 704b having a braided reinforcement layer (or coiled reinforcement layer) and a tapered tip. In particular, the braided reinforcement layer (or coiled reinforcement layer) of catheter tip 708b extends to the distal-most end. In yet another embodiment, catheter tip 708c includes a catheter wall 704c having a braided reinforcement layer (or coiled reinforcement layer) and a constant diameter wall with a rounded tip.

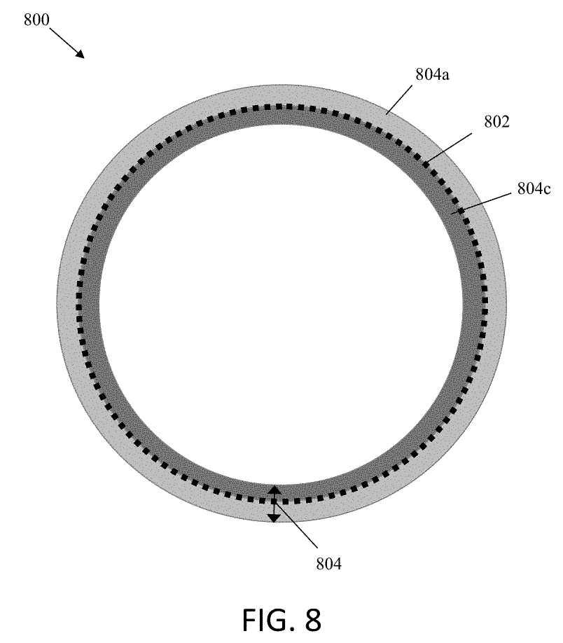

FIG. 8 shows a cross-sectional view of a catheter shaft 800 having two polymer layers and a braided reinforcement layer 802. In particular, the catheter shaft 800 includes a catheter wall 804 having an outer layer 804a, the braided reinforcement layer 802, and an inner layer 804c. As shown in FIG. 8, the braided reinforcement layer 802 is disposed between the outer layer 804a and the inner layer 804c. In another embodiment, the reinforcement layer 802 may be a coiled reinforcement layer as described with respect to FIGS. 4A and 4B or a combination of coiled and braided reinforcement layers as described with respect to FIGS. 5A and 5B.

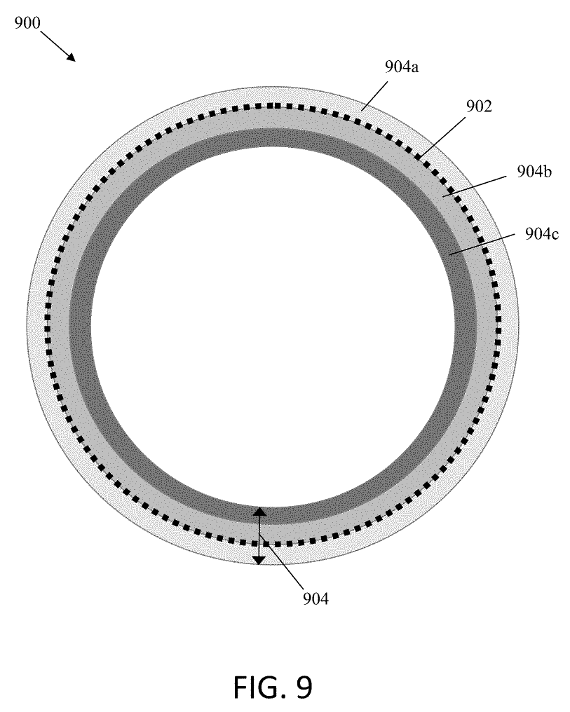

FIG. 9 shows a cross-sectional view of a catheter shaft 900 having three polymer layers and a braided reinforcement layer 902. In particular, the catheter shaft 900 includes a catheter wall 904 having an outer layer 904a, the braided reinforcement layer 902, a middle layer 904b, and an inner layer 904c. As shown in FIG. 9, the braided reinforcement layer 902 is disposed between the outer layer 904a and the middle layer 904b. Alternatively, the reinforcement layer may be between the inner 904c and middle 904b layers. In yet another embodiment, the reinforcement layer may be a coiled reinforcement layer as described with respect to FIGS. 4A and 4B or a combination of coiled and braided reinforcement layers as described with respect to FIGS. 5A and 5B.

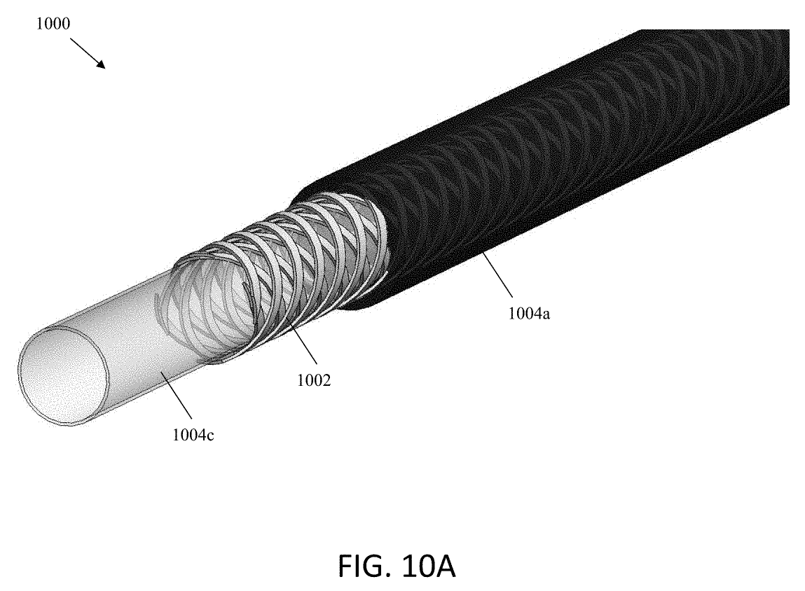

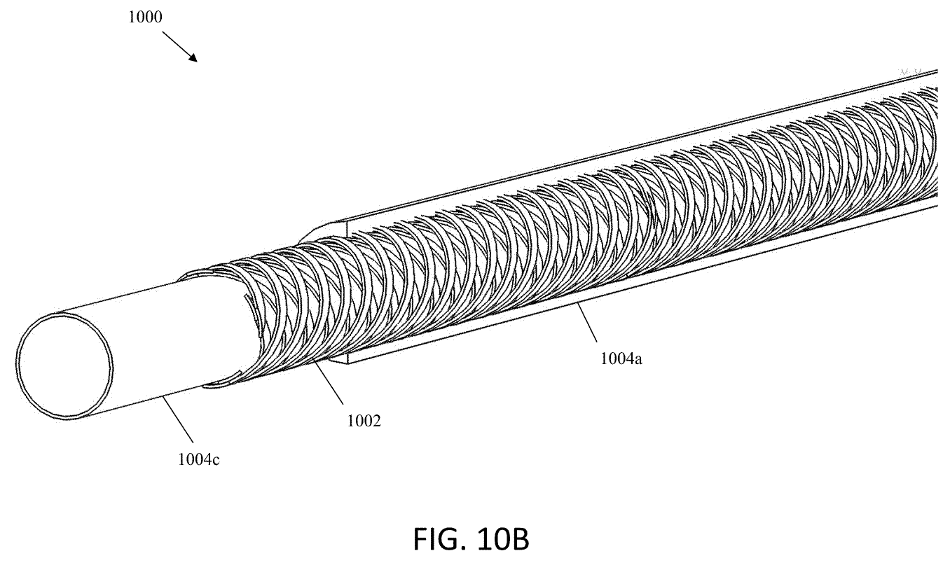

FIGS. 10A and 10B show a cutaway and cross-sectional view, respectively, of a catheter shaft 1000 having a braided reinforcement layer 1002. In particular, the catheter shaft 1000 includes a catheter wall having multiple layers: an outer layer 1004a, a braided reinforcement layer 1002, and an inner layer 1004d. In another embodiment, the catheter shaft may comprise one or more middle layers (not shown). The braided reinforcement layer 1002 may be embedded between the outer layer 1004a and the inner layer 1004c or may be disposed in the space between the outer layer 1004a and the middle layer 1004c. The coiled reinforcement layer may be similarly positioned between the layers.

The catheter shaft of the invention may be manufactured in a continuous (reel-to-reel) manufacturing process or a discrete manufacturing process using a mandrel. The continuous “reel-to-reel” method of manufacturing a catheter shaft may include forming an inner catheter wall layer around a mandrel. The mandrel may be made of a metal, such as, for example, stainless steel, titanium, or copper. To form an inner layer of polymer, the mandrel may be passed through a first polymer in a liquid state, such as liquid PTFE. The liquid first polymer may be cured by heating in, e.g., an oven. One or more optional middle layer(s) of polymer may be formed over the inner layer by passing the mandrel and hardened inner layer through a second polymer in a liquid state, such as, e.g., liquid PEBAX. The liquid second polymer may be cured by heating in, e.g., an oven. A reinforcement layer may be wound or woven over the first layer of polymer by a winding/braiding machine. In the embodiment wherein one or more middle layer(s) are formed, the reinforcement layer may be wound or woven over any one of the one or more middle layer(s). For example, a coil may be wound around the first, inner layer (or one of the optional middle layers) and/or a braid may be woven around that layer. The mandrel, the first layer of polymer, the optional one or more middle layer(s) of polymer, and the reinforcement layer may be passed through a third layer of polymer in a liquid state, such as, e.g., liquid PEBAX. The liquid third polymer may be cured by heating in, e.g., an oven. In certain embodiments, no first polymer (i.e., the inner layer having a low friction coefficient) is used and the resulting catheter shaft includes at least two polymer layers with a reinforcement layer therebetween.

In an alternative method of manufacturing a catheter shaft, the inner layer, one or more optional middle layer(s), and outer layer are formed from extruded tubes that are heat shrunk over one another. In particular, a first polymer tube (e.g., PTFE) may be extruded and positioned on the mandrel. The first polymer tube may be heated to thereby shrink the tube around the mandrel. One or more optional second polymer tube(s) (e.g., PEBAX) may be extruded and positioned on the mandrel and shrunk over the first polymer tube to form one or more of the optional middle layers. The optional second polymer tube may be heated to thereby shrink the tube in a similar fashion around the mandrel and first polymer tube. Similar to the continuous process, a reinforcement layer may be wound or woven over the first layer of polymer by a winding/braiding machine. In the embodiment wherein one or more middle layer(s) are formed, the reinforcement layer may be wound or woven over any one of the one or more middle layer(s). For example, a coil may be wound around the first polymer tube and/or a braid may be woven around the first polymer tube. A third polymer tube (e.g., PEBAX) may be extruded and positioned on the mandrel, first polymer tube, optional one or more middle tube(s), and reinforcement layer. The third polymer tube may be heated to thereby shrink the tube around the mandrel, first polymer tube, optional one or more middle tube (s), and reinforcement layer.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the invention disclosed herein. All references cited herein are incorporated by reference in their entirety and made part of this application.

1. A catheter shaft comprising a tubular wall defining a lumen, said wall comprising:
an outer layer, wherein the outer layer comprises a first polymer;
an inner layer, wherein the inner layer comprises a second polymer and is adjacent said outer layer; and
a reinforcement layer disposed between the outer layer and the inner layer, said reinforcement layer comprising a coiled and/or a braided structure.

2. The catheter shaft of claim 1, further comprising a middle layer comprising a third polymer.

3. The catheter shaft of claim 1, wherein the reinforcement layer comprises a metal selected from the group consisting of: stainless steel, titanium, gold, platinum-iridium, beryllium copper, silver, MP35, and nitinol.

4. The catheter shaft of claim 1, wherein the reinforcement layer comprises a polymer selected from the group consisting of: polyamide, nylon, polyurethane, poly-paraphenylene terephthalamide, poly-paraphenylene terephthalamide, liquid crystal polymer, and polyether ether ketone (PEEK).

5. The catheter shaft of claim 2, wherein the first polymer and the third polymer comprise the same polymer.

6. The catheter shaft of claim 2, wherein the first polymer and the third polymer comprise different polymers.

7. The catheter shaft of claim 5, wherein the first polymer and the third polymer comprise polyether block amide (PEBAX).

8. The catheter shaft of claim 1, wherein the second polymer is polytetrafluoroethylene (PTFE).

9. The catheter shaft of claim 1, wherein the braided reinforcement comprises a first picks per inch (PPI) along a first length of the catheter shaft.

10. The catheter shaft of claim 9, wherein the braided reinforcement comprises a second picks per inch (PPI) along a second length of the catheter shaft.

11. The catheter shaft of claim 10, wherein the first picks per inch (PPI) is greater than the second picks per inch (PPI).

12. The catheter shaft of claim 11, wherein the first length is distal to the second length.

13. The catheter shaft of claim 11, wherein the first length is proximal to the second length.

14. The catheter shaft of claim 9, wherein the first picks per inch (PPI) is between 20 and 150.

15. The catheter shaft of claim 1, wherein the coiled reinforcement comprises a first pitch space along a first length of the catheter shaft.

16. The catheter shaft of claim 15, wherein the coiled reinforcement comprises a second pitch space along a second length of the catheter shaft.

17. The catheter shaft of claim 16, wherein the first pitch space is greater than the second pitch space.

18. The catheter shaft of claim 17, wherein the first length is distal to the second length.

19. The catheter shaft of claim 17, wherein the first length is proximal to the second length.

20. The catheter shaft of claim 1, wherein the coiled reinforcement comprises a pitch space of 0.1 mm to 0.5 mm.

21. The catheter shaft of claim 1, wherein the catheter shaft comprises a constant diameter along the entire length of the catheter shaft.

22. The catheter shaft of claim 21, wherein the catheter shaft comprises a rounded end at a distal-most end of the catheter shaft.

23. The catheter shaft of claim 1, wherein the catheter shaft comprises a taper at a distal end of the catheter shaft.

24. The catheter shaft of claim 1, further comprising a catheter tip at a distal end of the catheter shaft, wherein the reinforcement layer terminates before a distal-most end the catheter tip.

25. The catheter shaft of claim 24, wherein the catheter tip has a length of about 2.5 mm to about 6 mm.

26. The catheter shaft of claim 1, wherein the catheter shaft comprises an outer diameter of 0.4 mm to 0.7 mm.

27. The catheter shaft of claim 1, wherein the catheter shaft defines a balloon region and a tip region that is distal to the balloon region, the catheter shaft further comprising a balloon coupled to the balloon region.

28. A percutaneous catheter assembly comprising:
the catheter shaft of claim 1; and
an inflatable balloon coupled to the catheter shaft at a distal end of the catheter shaft.

29. A method of manufacturing a catheter shaft comprising:
forming a first layer on a mandrel, wherein the first layer comprises a first polymer;
forming a reinforcement layer on the first layer;
forming a second layer over the reinforcement layer, wherein the second layer comprises a second polymer.

30. The method of claim 29, wherein the first polymeric layer comprises polytetrafluoroethylene (PTFE).

31. The method of claim 29, wherein the second polymeric layer comprises polyether block amide (PEBAX).

32. The method of claim 29, wherein the mandrel comprises copper.

33. The method of claim 29, wherein forming the first polymeric layer comprises:
passing the mandrel through a liquid state of the first polymer; and
heating the liquid first polymer to solidify the liquid first polymer.

34. The method of claim 33, wherein forming the reinforcement layer comprises:
winding a reinforcement material over the first polymeric layer.

35. The method of claim 34, wherein forming the second polymeric layer comprises:
passing the mandrel, first polymeric layer, and reinforcement layer through a liquid state of the second polymer; and
heating the liquid second polymer to solidify the liquid second polymer.

36. The method of claim 29, wherein forming the first polymeric layer comprises:
extruding a first tube comprising the first polymer; and
heating the first tube to shrink the first tube onto the mandrel.

37. The method of claim 36, wherein forming the reinforcement layer comprises:
winding a reinforcement material over the first polymeric layer.

38. The method of claim 37, wherein forming the second polymeric layer comprises:
extruding a second tube comprising the second polymer; and
heating the second tube to shrink the second tube onto the first polymeric layer and reinforcement layer.
